(19) United States
(12) Patent Application Publication
(10) Pub. No.: US 2017/0042446 A1
(43) Pub. Date: Feb. 16, 2017

(54) BIOSIGNAL DETECTING APPARATUS AND METHOD THEREFOR

(71) Applicant: KOREA ADVANCED INSTITUTE OF SCIENCE AND TECHNOLOGY, Daejeon (KR)

(72) Inventors: Seong Ook Park, Daejeon (KR); Jun Seuk Suh, Daejeon (KR); Rao Shahid Aziz, Daejeon (KR)

(21) Appl. No.: 14/938,563

(22) Filed: Nov. 11, 2015

(30) Foreign Application Priority Data
Aug. 12, 2015 (KR) 10-2015-0114055

(51) Int. Cl. A61B 5/05; A61B 5/00; G01S 7/02

(52) U.S. Cl. CPC A61B 5/05; G01S 7/026; A61B 5/725; A61B 5/7203; A61B 5/7225

(57) ABSTRACT

A biosignal detecting apparatus and a method therefor are provided. The biosignal detecting apparatus includes a signal generator configured to generate a predetermined specific frequency signal, at least two transmit helix antennas configured to transmit circular polarizations according to the specific frequency signal to a subject, a receive helix antenna configured to receive a circular polarization including a biosignal reflected from the subject, a mixer configured to generate an intermediate frequency (IF) signal by mixing the received circular polarization with the specific frequency signal and a detecting unit configured to detect the biosignal of the subject from the IF signal.

### CROSS-REFERENCE TO RELATED APPLICATIONS

The entire contents of Korean Patent Application No. 10-2015-0114055 filed Aug. 12, 2015, in the Korean Intellectual Property Office, are hereby incorporated by reference.

### BACKGROUND

Embodiments of the inventive concepts described herein relate to technologies for detecting noninvasive biosignals, and more particularly, to a noninvasive biosignal detecting apparatus for detecting noninvasive biosignals, such as breathing signals and heart rates which have periodic movement of skins and muscles near a heart of a subject, using circularly polarized helix antennas and a method therefor.

It has been important to systematically monitor noninvasive biosignals, such as breathing signals and heart rates, in a medical field. For a long time, non-contact monitoring for heart and breathing capabilities of patients and old people according to microwave Doppler radars is very invaluable to remote detection and diagnosis of syndrome risks and heart attacks in medicine and telemedicine.

A primary task in this research field is to extract accurate information in a situation where many noises, clutters, and body motion interference are present. An initial system is big, heavy, and expensive. However, the initial system may be integrated into partial modules by recent technologies which use wireless systems. Doppler radars are used in remote health care, ubiquitous home networking, vehicle impact prevention, a microwave life-detection system which may seek people below earthquake rubbles or behind barriers, and the like. In a current medical trend, a sensing method using a remote sensing system is an important matter of interest.

A typical noninvasive biosignal measuring apparatus measures heart rates, breathing signals, and the like by attaching sensors to a subject such as a person or target. Since sensors are attached to the subject, movement of the subject is not free. Therefore, noises may be generated by movement of the subject.

There is a radar system which measures noninvasive biosignals within a specific range using radar without attaching sensors to a subject. The radar system transmits electromagnetic continuous signals and receives reflective signals from the subject. It is important to accurately and effectively detect biosignals, such as heart rates and breathing signals, which are reflected from the subject. This is because the reflected biosignals include noises due to movement of the subject or other causes. Therefore, it is important to remove noises to obtain accurate heart rates and accurate breathing signals.

Another method of detecting biosignals such as heart rates and breathing signals previously determines reference signals. The reference signals are preset for each of a detected heart rate and a detected breathing signal. Only noise signals may be obtained from obtained biosignals by removing the reference signals from the obtained biosignals. Also, heart rates and breathing signals may be detected by removing noise signals from biosignals.

An important factor of this method is that all frequency band signals should be included in reference signals. Since characteristics of heart rates and breathing signals are similar to each other, reference signals may not be preset. Also, because of the non-contact type, amplitude and phases of signals may be changed according to a distance between a radio frequency (RF) sensor and a subject. Therefore, changes in amplitude and phase should be able to be compensated.

Another method of detecting biosignals uses the concept of circular polarization in a single transmitter and a single receiver. There may be two patch antenna arrays including an antenna array for transmission and an antenna array for reception. In this case, the two patch antenna arrays may have different circular polarizations. For example, a transmitter may have a left-hand circular polarization (LHCP), and a receiver may have a right-hand circular polarization (RHCP).

Following a principle in which the sensing of circular polarization rotation is reversed when a reflective surface is fully conducted, LHCP is reflected as RHCP on skins and muscles near a heart of a person, which have a high dielectric constant and high conductivity. A radar system may be used to detect biosignals, such as heart rates and breathing signals near a heart of a person, in a noninvasive manner.

However, when a subject is distant from a Doppler radar system, a transmit signal and a receive signal may be very weak. Also, it is difficult to measure heart rates and breathing signals through a bistatic radar system which uses a transmit antenna and a receive antenna. The test setup of the bistatic radar system is for a subject who sits on a chair. There may be a major problem in alignment of transmitters and receivers.

Therefore, there is a need for an apparatus which may more accurately and effectively detect biosignals, such as heart rates and breathing signals, using a radar system.

### SUMMARY

Embodiments of the inventive concepts provide a noninvasive biosignal detecting apparatus which may improve detection accuracy of a noninvasive biosignal using a circularly polarized helix antenna.

One aspect of embodiments of the inventive concept is directed to provide a biosignal detecting apparatus. The biosignal detecting apparatus may include a signal generator configured to generate a predetermined specific frequency signal, at least two transmit helix antennas configured to transmit circular polarizations according to the specific frequency signal to a subject, a receive helix antenna configured to receive a circular polarization including a biosignal reflected from the subject, a mixer configured to generate an intermediate frequency (IF) signal by mixing the received circular polarization with the specific frequency signal, and a detecting unit configured to detect the biosignal of the subject from the IF signal.

The at least two transmit helix antennas may transmit left-hand circular polarizations (LHCPs). The receive helix antenna may receive a right-hand circular polarization (RHCP).

The receive helix antenna may be located in a predetermined distance between the at least two transmit helix antennas.

The predetermined distance may be determined by a wavelength of the specific frequency signal.

The at least two transmit helix antennas may transmit circular polarizations having a phase difference of 180 degrees.

The biosignal detecting apparatus may further include an amplifier configured to amplify the specific frequency signal, a first power divider configured to divide the amplified specific frequency signal into two signals, and a second power divider configured to receive the divided one specific frequency signal, to divide the received signal into two signals to have a phase difference of 180 degrees, and to provide the divided two signals to the at least two transmit helix antennas, respectively.

The detecting unit may include a first amplifier configured to amplify the IF signal, a remover configured to remove direct current (DC) components from the amplified IF signal, a second amplifier configured to amplify the IF signal in which the DC components are removed, a low pass filter (LPF) configured to low pass filter the signal amplified by the second amplifier, an analog to digital converter (ADC) configured to convert an output signal of the LPF into a digital signal, and a biosignal detector configured to detect the biosignal of the subject using the converted digital signal.

Another aspect of embodiments of the inventive concept is directed to provide a biosignal detecting method. The biosignal detecting method may include generating a predetermined specific frequency signal, transmitting circular polarizations, according to the specific frequency signal to a subject, using at least two transmit helix antennas, receiving a circular polarization, including a biosignal reflected from the subject, using a receive helix antenna, generating an intermediate frequency (IF) signal by mixing the received circular polarization with the specific frequency signal, and detecting the biosignal of the subject from the IF signal.

The transmitting of the circular polarizations may include transmitting left-hand circular polarizations (LHCPs) using the at least two transmit helix antennas. The receiving of the circular polarization may include receiving a right-hand circular polarization (RHCP) using the receive helix antenna.

The receive helix antenna may be located in a distance between the at least two transmit helix antennas, which is determined by a wavelength of the specific frequency signal.

The transmitting of the circular polarizations may include transmitting circular polarizations, having a phase difference of 180 degrees, using the at least two transmit helix antennas.

The biosignal detecting method may further include amplifying the specific frequency signal, dividing the amplified specific frequency signal into two signals, and receiving the divided one specific frequency signal, dividing the received signal into two signals to have a phase difference of 180 degrees, and providing the divided two signals to the at least two transmit helix antennas, respectively. The transmitting of the circular polarizations may include transmitting circular polarizations, according to the signals divided to have the phase difference of 180 degrees, to the subject.

The detecting of the bio signal may include amplifying the IF signal, removing direct current (DC) components from the amplified IF signal, amplifying the IF signal in which the DC components are removed and low pass filtering the amplified IF signal, converting the low pass filtered signal into a digital signal, and detecting the biosignal of the subject using the converted digital signal.

### BRIEF DESCRIPTION OF THE FIGURES

The above and other objects and features will become apparent from the following description with reference to the following figures, wherein like reference numerals refer to like parts throughout the various figures unless otherwise specified, and wherein

FIG. 1 is a block diagram illustrating a configuration of a biosignal detecting apparatus according to an exemplary embodiment of the inventive concept;

FIG. 2 is a block diagram illustrating a detailed configuration of a biosignal detecting apparatus according to an exemplary embodiment of the inventive concept;

FIG. 3 is a drawing illustrating measured return losses of circularly polarized helix antennas according to an exemplary embodiment of the inventive concept;

FIG. 4 is a drawing illustrating radiation patterns of circularly polarized helix antennas according to an exemplary embodiment of the inventive concept;

FIG. 5 is a drawing illustrating measured gains of circularly polarized helix antennas and a gain of a standard horn antenna according to an exemplary embodiment of the inventive concept;

FIG. 6 is a flowchart illustrating an operation of a biosignal detecting method according to an exemplary embodiment of the inventive concept; and

FIG. 7 is a flowchart illustrating a detailed operation of step S360 shown in FIG. 6 according to an exemplary embodiment of the inventive concept.

### DETAILED DESCRIPTION

Embodiments will be described in detail with reference to the accompanying drawings. The inventive concept, however, may be embodied in various different forms, and should not be construed as being limited only to the illustrated embodiments. Rather, these embodiments are provided as examples so that this disclosure will be thorough and complete, and will fully convey the concept of the inventive concept to those skilled in the art. Accordingly, known processes, elements, and techniques are not described with respect to some of the embodiments of the inventive concept. Unless otherwise noted, like reference numerals denote like elements throughout the attached drawings and written description, and thus descriptions will not be repeated. In the drawings, the sizes and relative sizes of layers and regions may be exaggerated for clarity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Also, the term “exemplary” is intended to refer to an example or illustration.

It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

Hereinafter, a description will be given in detail for exemplary embodiments of the inventive concept with reference to the accompanying drawings. However, the inventive concept is not limited by exemplary embodiments. Also, with respect to the descriptions of the drawings, like reference numerals refer to like elements.

According to exemplary embodiments of the inventive concept, a biosignal detecting apparatus accurately detects a noninvasive biosignal using a circularly polarized helix antenna. There is the subject matter that the biosignal detecting apparatus improves detection accuracy of a biosignal by transmitting left-hand circular polarizations (LHCPs), having a phase difference of 180 degrees, using at least two transmit helix antennas and receiving a right-hand circular polarization (RHCP) reflected from a subject using one receive helix antenna.

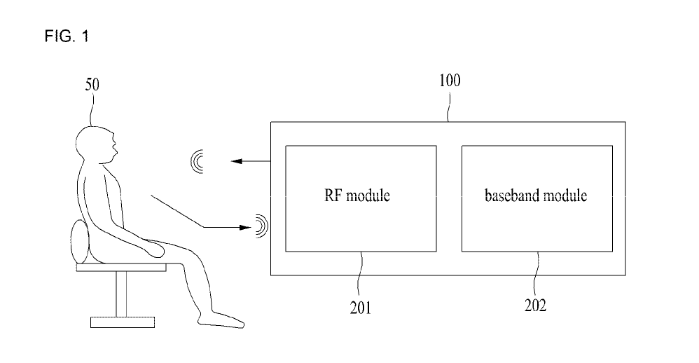

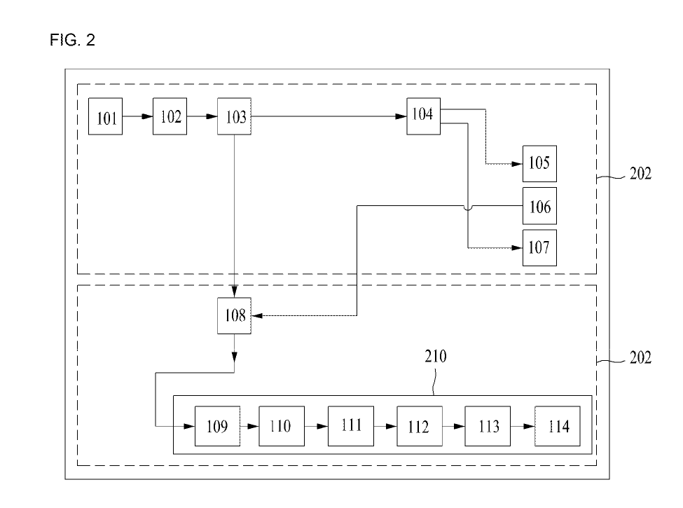

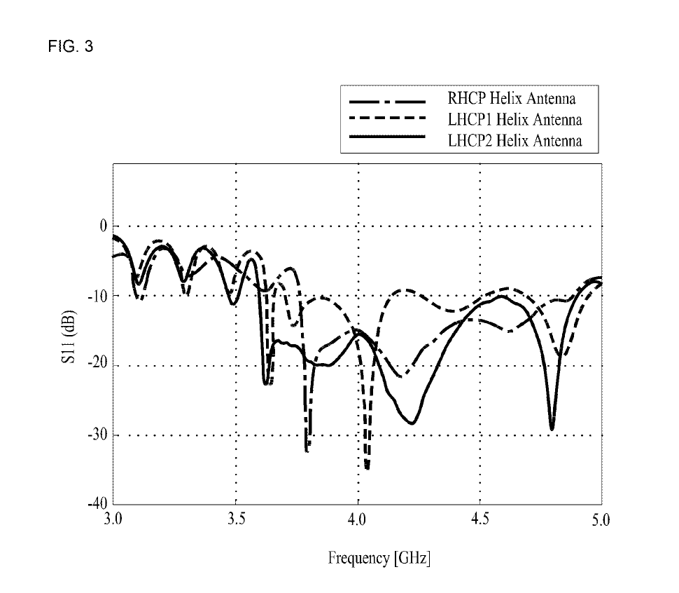

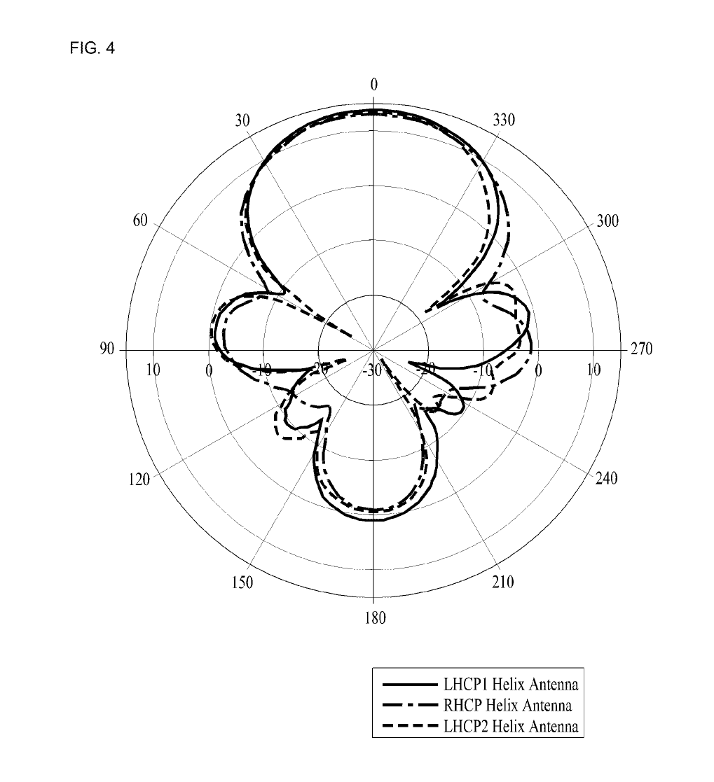

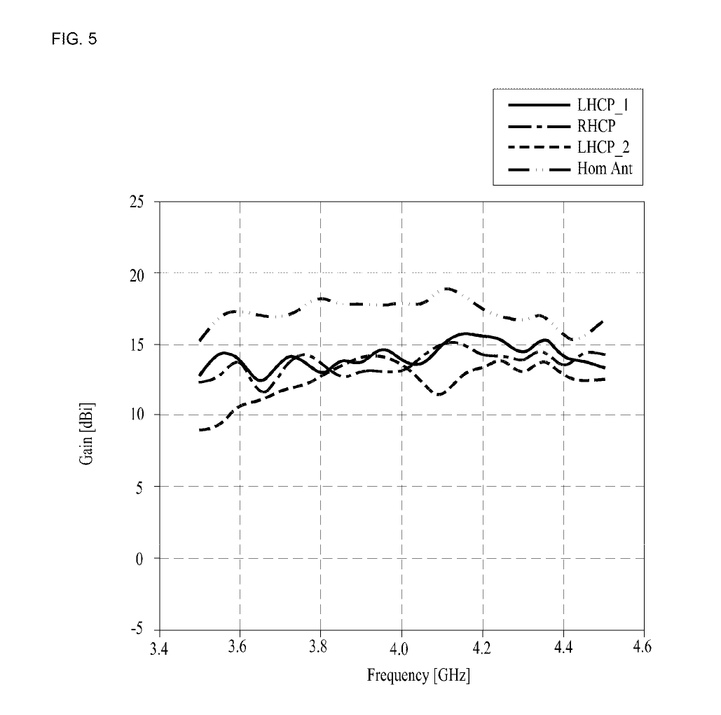

FIG. 1 is a block diagram illustrating a configuration of a biosignal detecting apparatus according to an exemplary embodiment of the inventive concept. FIG. 2 is a block diagram illustrating a detailed configuration of a biosignal detecting apparatus according to an exemplary embodiment of the inventive concept. FIG. 3 is a drawing illustrating measured return losses of circularly polarized helix antennas according to an exemplary embodiment of the inventive concept. FIG. 4 is a drawing illustrating radiation patterns of circularly polarized helix antennas according to an exemplary embodiment of the inventive concept. FIG. 5 is a drawing illustrating measured gains of circularly polarized helix antennas and a gain of a standard horn antenna according to an exemplary embodiment of the inventive concept.

Referring to FIGS. 1 and 2, a biosignal detecting apparatus 100 according to an exemplary embodiment of the inventive concept may include a radio frequency (RF) module 201 and a baseband module 202.

The RF module 201 may include a signal generator 101, an amplifier 102, a first power divider 103, a second power divider 104, a first transmit helix antenna 105, a second transmit helix antenna 107, and a receive helix antenna 106. The baseband module 202 may include a mixer 108 and a detecting unit 210. The detecting unit 210 may include a first amplifier 109, a remover 110, a second amplifier 111, a low pass filter (LPF) 112, an analog to digital converter (ADC) 113, and a biosignal detector 114.

The biosignal detecting apparatus 100 according to an exemplary embodiment of the inventive concept may accurately detect a biosignal, such as a heart rate and a breathing signal of a subject 50 by transmitting circular polarizations to the subject 50 using at least two transmit helix antennas, and receiving a circular polarization including a biosignal reflected from the subject 50, removing noises from the received circular polarization, and converting the signal in which the noises are removed into a digital signal.

Describing the RF module 201, the signal generator 101 may generate a signal of a predetermined specific frequency, for example, 4 GHz.

In this case, the signal generator 101 may include a local oscillator.

The amplifier 102 may amplify the specific frequency signal generated by the signal generator 101. The first power divider 103 may divide the specific frequency signal amplified by the amplifier 102 into two signals and may provide the two signals to the second power divider 104 and the mixer 108, respectively.

The second power divider 104 may divide a signal, that is, a specific frequency signal, received from the first power divider 103 into two signals having a phase difference of 180 degrees and may provide the two signals to the first transmit helix antenna 105 and the second transmit helix antenna 107, respectively.

The first transmit helix antenna 105 and the second transmit helix antenna 107 may transmit left-hand circular polarizations to the subject 50 according to the specific frequency signal having the phase difference of 180 degrees.

The receive helix antenna 106 may receive a reflective signal reflected from the subject 50.

In this case, the receive helix antenna 106 may receive a right-hand circular polarization (RHCP) reflected from the subject 50.

In other words, according to exemplary embodiments of the inventive concept, the first and second transmit helix antennas 105 and 107 and the receive helix antenna 106 may have different circular polarizations, respectively. This is because a reverse circular polarization rotation phenomenon is used. This phenomenon follows a principle in which the sensing of circular polarization rotation is reversed when a reflective surface is fully conducted. An LHCP may be reflected as an RHCP on skins and muscles near a heart of a person, which have a high dielectric constant and high conductivity.

The receive helix antenna 106 may be located between the first and second transmit helix antennas 105 and 107 used for transmission to accurately receive a biosignal from the subject 50. A distance between the first and second transmit helix antennas 105 and 107 and the receive helix antenna 106 may approximate or be determined by a wavelength A of a specific frequency signal.

Referring to FIGS. 3 and 4, return losses S11 of two transmit helix antennas LHCP1 and LHCP2 which transmit left-hand circular polarizations (LHCPs) and a receive helix antenna RHCP which receives a right-hand circular polarization (RCHP) may be shown in FIG. 3. Radiation patterns of the two transmit helix antennas LHCP1 and LHCP2 and the receive helix antenna RHCP may be shown in FIG. 4.

Referring to FIG. 5, measured gains of two transmit circularly polarized helix antennas LHCP_1 and LHCP_2, a measured gain of a receive circularly polarized helix antenna RHCP, and a gain of a horn antenna may be shown in FIG. 5.

As such, the biosignal detecting apparatus according to an exemplary embodiment of the inventive concept may include a high sensitive Doppler radar system which includes a transmit circularly polarized helix antenna and a receive circularly polarized helix antenna.

As described above, the RF module 201 according to an exemplary embodiment of the inventive concept may transmit a maximum transmit signal to the subject 50 by transmitting the circular polarizations, having the phase difference of 180 degrees, using the two transmit helix antennas 105 and 107. Therefore, the baseband module 202 may accurately detect a biosignal such as a heart rate and a breathing signal because receiving a reflective signal reflected from the subject 50 as a maximum signal.

Describing the baseband module 202, the mixer 108 may generate an intermediate frequency (IF) signal by mixing the specific frequency signal divided by the first power divider 103 with the circular polarization received through the receive helix antenna 106 to.

Herein, the mixer 108 is described as being included in the baseband module 202. However, in some cases, the mixer 108 may be included in the RF module 201.

The detecting unit 210 may remove a noise from the IF signal and may detect a biosignal of the subject 50 using the IF signal in which the noise is removed.

Specifically, the first amplifier 109 may amplify the IF signal. The remover 110 may remove direct current (DC) components from the amplified IF signal to prevent a DC offset.

In this case, the first amplifier 109 may be an instrumentation amplifier. The remover 110 may be a high pass filter (HPF).

The second amplifier 111 may amplify the IF signal in which the DC components are removed. The LPF 112 may low pass filter a signal output from the second amplifier 111. In other words, the LPF 112 may filter a high frequency signal, included in the signal output from the second amplifier 111, through low pass filtering.

The ADC 113 may convert a signal output from the LPF 112 into a digital signal. The biosignal detector 114 may detect a biosignal of the subject 50 according to the converted digital signal.

In this case, the biosignal detector 114 may be a notebook or a computer. The detected biosignal may be displayed on a screen through a display means (not shown).

As described above, the baseband module 202 may obtain only a desired signal, in which a noise is removed, by filtering and amplifying a biosignal received from a Doppler radar system and may convert the obtained signal into a digital signal.

Therefore, the biosignal detecting apparatus according to an exemplary embodiment of the inventive concept may detect a biosignal, such as a heart rate and a breathing signal which have periodic movement of muscles and skins near a heart of a subject, in a noninvasive manner using the Doppler radar system.

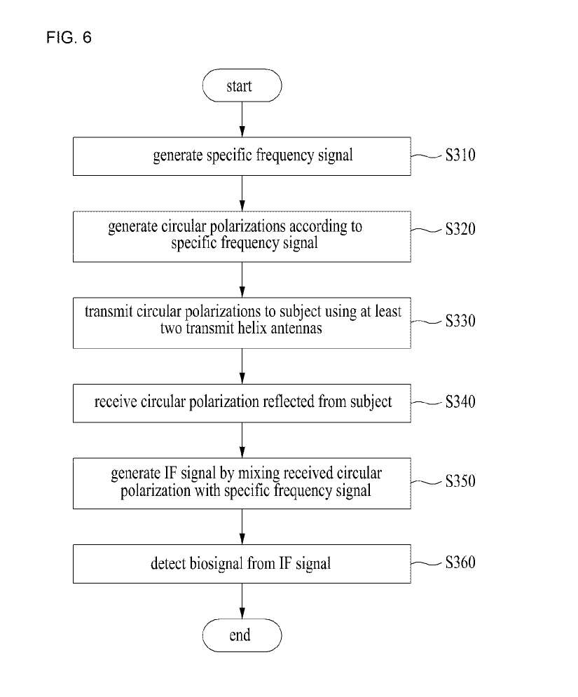

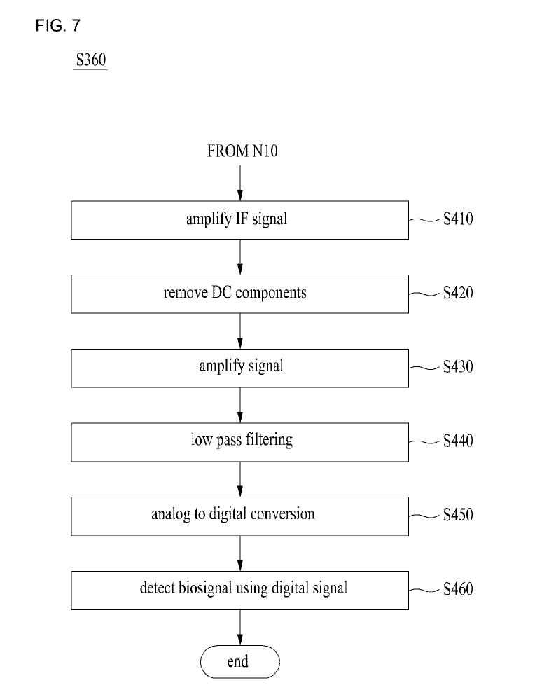

FIG. 6 is a flowchart illustrating an operation of a biosignal detecting method according to an exemplary embodiment of the inventive concept. FIG. 6 is a flowchart illustrating an operation of a biosignal detecting apparatus of FIGS. 1 and 2. FIG. 7 is a flowchart illustrating a detailed operation of step S360 shown in FIG. 6 according to an exemplary embodiment of the inventive concept.

Referring to FIG. 6, in the biosignal detecting method according to an exemplary embodiment of the inventive concept, in step S310, the biosignal detecting apparatus may generate a predetermined specific frequency signal using its local oscillator.

After generating the specific frequency signal, for example, a signal of 4 GHz in step S310, in step S320, the biosignal detecting apparatus may generate circular polarizations according to the generated specific frequency signal. In step S330, the biosignal detecting apparatus may transmit the generated circular polarizations to a subject using at least two transmit helix antennas.

Herein, in steps S320 and S330, the biosignal detecting apparatus may amplify the specific frequency signal generated in step S310, may divide the amplified specific frequency signal into two signals, may divide the divided one specific frequency signal into two signals to have a phase difference of 180 degrees, and provides the divided two signals to the two transmit helix antennas, respectively. Therefore, each of the two transmit helix antennas may transmit a circular polarization, according to the signals divided to have the phase difference of 180 degrees, to the subject.

In this case, each of the two transmit helix antennas may transmit a left-hand circular polarization (LHCH) having the phase difference of 180 degrees to the subject.

After transmitting the circular polarizations to the subject through the two transmit helix antennas in step S330, in step S340, the biosignal detecting apparatus may receive a circular polarization, including a biosignal reflected from the subject, using the receive helix antenna.

In this case, the receive helix antenna may receive a right-hand circular polarization (RHCP) reflected from the subject. The receive helix antenna may be located between the two transmit helix antennas used for transmission to accurately receive a biosignal from the subject. A distance between the two transmit helix antennas and the receive helix antenna may approximate and be determined by a wavelength A of the specific frequency signal.

After receiving the circular polarization reflected from the subject in step S340, in step S350, the biosignal detecting apparatus may generate an IF signal by mixing the received circular polarization with the specific frequency signal generated in step S310. In step S360, the biosignal detecting apparatus may detect a biosignal from the generated IF signal.

Herein, step S360 of detecting the biosignal may include, as shown in FIG. 7, steps S410 to S460. In step S410, the biosignal detecting apparatus may amplify the IF signal generated in step S360. In step S420, the biosignal detecting apparatus may remove DC components from the amplified IF signal to prevent a DC offset.

Herein, in step S410, the biosignal detecting apparatus may amplify the IF signal using an instrumentation amplifier. In step S420, the biosignal detecting apparatus may remove the DC components from the amplified IF signal using an HPF.

In step S430, the biosignal detecting apparatus may amplify the IF signal in which the DC components are removed. In step S440, the biosignal detecting apparatus may low pass filter the amplified signal. Therefore, the biosignal detecting apparatus may filter a high frequency signal included in the IF signal in which the DC components are removed.

In step S450, the biosignal detecting apparatus may convert the low pass filtered signal in step S440 into a digital signal. In step S460, the biosignal detecting apparatus may detect a biosignal of the subject according to the converted digital signal.

The biosignal detected in step S460 may be displayed on a screen through a display means such as a notebook and a computer.

According to exemplary embodiments of the inventive concept, the biosignal detecting apparatus may accurately detect a noninvasive biosignal using a circularly polarized helix antenna.

According to exemplary embodiments of the inventive concept, the biosignal detecting apparatus may accurately detect a biosignal by transmitting LHCPs, having a phase difference of 180 degrees, to a subject using at least two transmit helix antennas and receiving an RHCP reflected from the subject using one receive helix antenna.

The foregoing systems or devices may be realized by hardware elements, software elements and/or combinations thereof. For example, the systems, devices and components illustrated in the exemplary embodiments of the inventive concept may be implemented in one or more general-use computers or special-purpose computers, such as a processor, a controller, an arithmetic logic unit (ALU), a digital signal processor, a microcomputer, a field programmable array (FPA), a programmable logic unit (FLU), a microprocessor or any device which may execute instructions and respond. A processing unit may implement an operating system (OS) or one or software applications running on the OS. Further, the processing unit may access, store, manipulate, process and generate data in response to execution of software. It will be understood by those skilled in the art that although a single processing unit may be illustrated for convenience of understanding, the processing unit may include a plurality of processing elements and/or a plurality of types of processing elements. For example, the processing unit may include a plurality of processors or one processor and one controller. Also, the processing unit may have a different processing configuration, such as a parallel processor.

Software may include computer programs, codes, instructions or one or more combinations thereof and configure a processing unit to operate in a desired manner or independently or collectively control the processing unit. Software and/or data may be permanently or temporarily embodied in any type of machine, components, physical equipment, virtual equipment, computer storage media or units or transmitted signal waves so as to be interpreted by the processing unit or to provide instructions or data to the processing unit. Software may be dispersed throughout computer systems connected via networks and be stored or executed in a dispersion manner. Software and data may be recorded in one or more computer-readable storage media.

The methods according to the above-described exemplary embodiments of the inventive concept may be implemented with program instructions which may be executed through various computer means and may be recorded in computer-readable media. The media may also include, alone or in combination with the program instructions, data files, data structures, and the like. The program instructions recorded in the media may be designed and configured specially for the exemplary embodiments of the inventive concept or be known and available to those skilled in computer software. Examples of computer-readable media include magnetic media such as hard disks, floppy disks, and magnetic tape; optical media such as compact disc-read only memory (CD-ROM) disks and digital versatile discs (DVDs); magneto-optical media such as floptical disks; and hardware devices that are specially configured to store and perform program instructions, such as read-only memory (ROM), random access memory (RAM), flash memory, and the like. Examples of program instructions include both machine codes, such as produced by a compiler, and higher level codes that may be executed by the computer using an interpreter. The described hardware devices may be configured to act as one or more software modules to perform the operations of the above-described exemplary embodiments of the inventive concept, or vice versa.

While a few exemplary embodiments have been shown and described with reference to the accompanying drawings, it will be apparent to those skilled in the art that various modifications and variations can be made from the foregoing descriptions. For example, adequate effects may be achieved even if the foregoing processes and methods are carried out in different order than described above, and/or the aforementioned elements, such as systems, structures, devices, or circuits, are combined or coupled in different forms and modes than as described above or be substituted or switched with other components or equivalents.

Therefore, other implements, other embodiments, and equivalents to claims are within the scope of the following claims.

What is claimed is:

1. A biosignal detecting apparatus comprising:
a signal generator configured to generate a predetermined specific frequency signal;
at least two transmit helix antennas configured to transmit circular polarizations according to the specific frequency signal to a subject;
a receive helix antenna configured to receive a circular polarization including a biosignal reflected from the subject;
a mixer configured to generate an intermediate frequency (IF) signal by mixing the received circular polarization with the specific frequency signal; and
a detecting unit configured to detect the biosignal of the subject from the IF signal.

2. The biosignal detecting apparatus of claim 1, wherein the at least two transmit helix antennas transmit left-hand circular polarizations (LHCPs), and
wherein the receive helix antenna receives a right-hand circular polarization (RHCP).

3. The biosignal detecting apparatus of claim 1, wherein the receive helix antenna is located in a predetermined distance between the at least two transmit helix antennas.

4. The biosignal detecting apparatus of claim 3, wherein the predetermined distance is determined by a wavelength of the specific frequency signal.

5. The biosignal detecting apparatus of claim 1, wherein the at least two transmit helix antennas transmit circular polarizations having a phase difference of 180 degrees.

6. The biosignal detecting apparatus of claim 1, further comprising:
an amplifier configured to amplify the specific frequency signal;
a first power divider configured to divide the amplified specific frequency signal into two signals; and
a second power divider configured to receive the divided one specific frequency signal, to divide the received signal into two signals to have a phase difference of 180 degrees, and to provide the divided two signals to the at least two transmit helix antennas, respectively.

7. The biosignal detecting apparatus of claim 1, wherein the detecting unit comprises:
a first amplifier configured to amplify the IF signal;
a remover configured to remove direct current (DC) components from the amplified IF signal;
a second amplifier configured to amplify the IF signal in which the DC components are removed;
a low pass filter (LPF) configured to low pass filter the signal amplified by the second amplifier;
an analog to digital converter (ADC) configured to convert an output signal of the LPF into a digital signal; and
a biosignal detector configured to detect the biosignal of the subject using the converted digital signal.

8. A biosignal detecting method comprising:
generating a predetermined specific frequency signal;
transmitting circular polarizations, according to the specific frequency signal to a subject, using at least two transmit helix antennas;
receiving a circular polarization, including a biosignal reflected from the subject, using a receive helix antenna;
generating an intermediate frequency (IF) signal by mixing the received circular polarization with the specific frequency signal; and
detecting the biosignal of the subject from the IF signal.

9. The biosignal detecting method of claim 8, wherein the transmitting of the circular polarizations comprises:
transmitting left-hand circular polarizations (LHCPs) using the at least two transmit helix antennas, and
wherein the receiving of the circular polarization comprises:
receiving a right-hand circular polarization (RHCP) using the receive helix antenna.

10. The biosignal detecting method of claim 8, wherein the receive helix antenna is located in a distance between the at least two transmit helix antennas, which is determined by a wavelength of the specific frequency signal.

11. The biosignal detecting method of claim 8, wherein the transmitting of the circular polarizations comprises:
transmitting circular polarizations, having a phase difference of 180 degrees, using the at least two transmit helix antennas.

12. The biosignal detecting method of claim 8, further comprising:
amplifying the specific frequency signal;
dividing the amplified specific frequency signal into two signals; and
receiving the divided one specific frequency signal, dividing the received signal into two signals to have a phase difference of 180 degrees, and providing the divided two signals to the at least two transmit helix antennas, respectively,
wherein the transmitting of the circular polarizations comprises:
transmitting circular polarizations, according to the signals divided to have the phase difference of 180 degrees, to the subject.

13. The biosignal detecting method of claim 8, wherein the detecting of the bio signal comprises:
amplifying the IF signal;
removing direct current (DC) components from the amplified IF signal;
amplifying the IF signal in which the DC components are removed and low pass filtering the amplified IF signal;
converting the low pass filtered signal into a digital signal; and
detecting the biosignal of the subject using the converted digital signal.
